(19) United States
(12) Patent Application Publication
(10) Pub. No.: US 2025/0002443 A1
(43) Pub. Date: Jan. 2, 2025

(54) PROCESS FOR LOW BYPRODUCT FORMATION OF METHYL METHACRYLATE FROM AN OXIDATIVE ESTERIFICATION REACTOR

(71) Applicant: Rohm and Haas Company, Collegeville, PA (US)

(72) Inventor: Kirk W. Limbach, Dresher, PA (US)

(21) Appl. No.: 18/698,872

(22) PCT Filed: Oct. 5, 2022
(86) PCT No.: PCT/US2022/045729

§ 371 Date: Apr. 5, 2024

Related U.S. Application Data
(63) Continuation of application No. 63/253,566, filed on Oct. 8, 2021.

(51) Int. Cl. C07C 67/39; B01J 23/52

(52) U.S. Cl. CPC C07C 67/39; B01J 23/52

(57) ABSTRACT

A process for producing methyl methacrylate by oxidative esterification is disclosed. The process comprises reacting methanol and methacrolein in a reactor system comprising an oxygen-containing gas and a heterogeneous noble metal-containing catalyst to produce methyl methacrylate. No Arrhenius base is added to the reactor system.

### FIELD OF THE INVENTION

The invention relates to a method for the production of methyl methacrylate via the oxidative esterification of methacrolein and methanol using a heterogeneous catalyst.

### BACKGROUND OF THE INVENTION

The conversion of an aldehyde and alcohol in the presence of oxygen to a carboxylic ester via oxidative esterification, and in particular the conversion of methacrolein and methanol in the presence of oxygen to methyl methacrylate, has been known for many years. For example, U.S. Pat. No. 4,249,019 discloses the use of a palladium (Pd)—lead (Pb) catalyst and other catalysts for this purpose.

Typical process configurations have included slurry catalyst bubble column reactors and slurry catalyst continuous stirred tank reactors (CSTR). Slurry type reactors for this chemistry typically use a catalyst of less than 200 μm size, and U.S. Pat. No. 6,228,800 discloses the use of an egg-shell type catalyst of less than 200 μm size for slurry reactions. Issues with the use of slurry catalysts stem from catalyst attrition which may limit the life of the catalyst and make filtration of the product stream difficult. According to CN1931824, these problems can be addressed through the use of a larger size catalyst charged to a fixed bed reactor. However, as noted in U.S. Patent Application Publication No. 2016/0251301, the use of larger catalyst particles leads to a reduced space-time yield and other potential disadvantages.

Fixed bed technology with larger catalyst particles has been implemented in U.S. U.S. Pat. No. 4,520,125, which discloses the use of a 4 mm diameter catalyst in a fixed bed system. The reactor feed in that case was relatively dilute, as it is in more recent discussions of fixed bed technology for this chemistry such as U.S. Patent Application Publication No. 2016/0251301 and U.S. Patent Application Publication No. 2016/0280628.

In commercial production facilities, the oxidative esterification reactors are followed by a separation section consisting of distillation columns to purify the product and recycle dewatered and otherwise purified unreacted reactants (see, e.g., U.S. Pat. No. 5,969,178) where the product and recycle often constitute the majority of the product stream. In part, this is because methanol is typically provided to the oxidative esterification reactor in excess to maximize the conversion of valuable methacrolein (see, e.g., U.S. Pat. No. 7,326,806).

Feed concentration of methacrolein into the oxidative esterification reactor varies in the literature from very low (see, e.g., U.S. Pat. No. 5,892,102) to around 35 wt % (see, e.g., U.S. Pat. No. 8,461,373). Methanol is typically the major constituent of the feed and the recycle stream that returns to the oxidative esterification reactor from the downstream separations section.

Catalysts for this chemistry have included various noble metals such as palladium-based catalysts including palladium-lead catalyst (see, e.g., U.S. Pat. No. 4,249,019) and gold-based or gold-containing catalysts (see, e.g., U.S. Pat. Nos. 7,326,806 and 8,461,373).

It is desirable to maximize selectivity and reduce the formation of all byproducts. In particular, byproduct methyl isobutyrate (MIB) is critical to reduce because it is difficult to separate from the product MMA and is undesirable in the product.

### SUMMARY OF THE INVENTION

The present invention is directed to a process for producing methyl methacrylate by oxidative esterification, the process comprising reacting methanol and methacrolein in a reactor system comprising an oxygen-containing gas and a heterogeneous noble metal-containing catalyst to produce methyl methacrylate, wherein no Arrhenius base is added to the reactor system.

### DETAILED DESCRIPTION OF THE INVENTION

All percentage compositions are weight percentages (wt %), and all temperatures are in ° C., unless otherwise indicated. Averages are arithmetic averages unless otherwise indicated. An “average concentration” is the arithmetic average of the concentration entering a region and the concentration exiting the region, where the region is an individual reactor, a reactor system, or a zone within a reactor or reactor system. An “average ratio” is the ratio of the average concentration of one component relative to the average concentration of another component. For example, the average ratio of methanol to methacrolein in a reactor system is calculated by dividing the average concentration of methanol entering and exiting the reactor system by the average concentration of methacrolein entering and exiting the reactor system.

A noble metal is any of gold, platinum, iridium, osmium, silver, palladium, rhodium and ruthenium. More than one noble metal may be present in the catalyst, in which case the limits apply to the total of all noble metals.

The “catalyst center” is the centroid of the catalyst particle, i.e., the mean position of all points in all coordinate directions. A diameter is any linear dimension passing through the catalyst center and the average diameter is the arithmetic mean of all possible diameters. The aspect ratio is the ratio of the longest to the shortest diameters.

A reactor system refers to one or more reactors where a designated reaction takes place. For example, the oxidative esterification of methacrolein to produce methyl methacrylate may be the designated reaction that takes place in the reactor system. The reactor system may comprise a single reactor or a plurality of reactors. Additionally, the reactor system may be subdivided into multiple zones, i.e., a multizone reactor system. Zones may be defined by physical separation, such as by walls or barriers that define separate areas, or by differences in the reaction conditions, such as, for example, pressure, temperature, composition or concentration of the catalyst, reactants, or other reaction components such as inert materials, pH modifiers, etc. For example, the reactor system may comprise a single reactor comprising a single zone, a single reactor comprising multiple zones, multiple reactors comprising a single zone in each reactor, multiple reactors where one or more reactors has a single zone and one or more reactors that comprise multiple zones, or multiple reactors each comprising multiple zones. By definition, a reactor system comprising multiple reactors would be considered a multizone reactor system. An example of a multizone reactor may be a continuous tubular reactor comprising multiple zones, including one or more mixing zones, a cooling zone, and one or more catalyst zones where the reaction takes place. Another example of a multizone single reactor may be a stirred bed reactor comprising internal walls containing the catalyst that defines a catalyst zone through which liquid reactants are circulated, and a feed/removal zone outside of the catalyst zone where the reactants enter the reactor and products exit the reactor. When referring to the average concentration or any ratio of the reactor system, the average concentration or ratio is calculated based on what enters the reactor system and what exits the reactor system.

The reactor system may comprise a reactor configured as a fluidized bed reactor, a fixed bed reactor, a trickle bed reactor, a packed bubble column reactor, or a stirred bed reactor. Preferably, the reactor system comprises a packed bubble column reactor.

The catalyst may be present in the form of a slurry or a fixed bed depending on the reactor in which the catalyst is present. For example, a slurry catalyst can be used in a stirred bed reactor or a fluidized bed reactor, whereas a fixed bed catalyst can be used in a fixed bed reactor, trickle bed reactor, or a packed bubble column reactor. Preferably, the catalyst is in the form of a fixed bed reactor.

The size of the catalyst can be selected based on the type of reactor. For example, a slurry catalyst may have an average particle diameter less than 200 μm, such as, for example, from 10 μm to 200 μm. A fixed bed catalyst may have an average particle diameter 200 μm or greater, such as, for example, from 200 μm to 30 mm. Preferably, the average diameter of the catalyst particle is at least 60 μm, preferably at least 100 μm, preferably at least 200 μm, preferably at least 300 μm, preferably at least 400 μm, preferably at least 500 μm, preferably at least 600 μm, preferably at least 700 μm, preferably at least 800 μm; preferably no more than 30 mm, preferably no more than 20 mm, preferably no more than 10 mm, preferably no more than 5 mm, preferably no more than 4 mm, preferably no more than 3 mm.

The noble metal-containing catalyst comprises particles of a noble metal. Preferably, the noble metal comprises palladium or gold, and more preferably the noble metal comprises gold.

The particles of a noble metal preferably have an average diameter of less than 15 nm, preferably less than 12 nm, more preferably less than 10 nm, and even more preferably less than 8 nm. The standard deviation of the average diameter of the noble metal particles is +/−5 nm, preferably +/−2.5 nm, and more preferably +/−2 nm. As used herein, the standard deviation is calculated by the following equation:

$$standard deviation = \sqrt{\frac{{\sum }_{i = 1}^{n} \vert x - \overline{x} \vert}{n}}$$

where x is the size of each particle, x is the mean of the n number of particles, and n is at least 500.

Preferably, the noble metal-containing catalyst further comprises titanium-containing particles.

The titanium-containing particles may comprise elemental titanium or a titanium oxide, TiOₓ. Preferably, the titanium-containing particles comprise a titanium oxide.

The titanium-containing particles preferably have an average diameter of less than 5 times the average diameter of the noble metal-containing particles, more preferably an average diameter of less than 4 times the average diameter of the noble metal-containing particles, even more preferably an average particle diameter of less than 3 times the average diameter of the noble metal-containing particles, still more preferably an average particle diameter of less than 2 times the average diameter of the noble metal-containing particles, and yet more preferably an average particle diameter of less than 1.5 times the average diameter of the noble metal-containing particles.

The amount by weight of the noble metal-containing particles with respect to the amount of the titanium-containing particles may range from 1:1 to 1:20. Preferably, the weight ratio of noble metal-containing particles to titanium-containing particles ranges from 1:2 to 1:15, more preferably from 1:3 to 1:10, even more preferably from 1:4 to 1:9, and still more preferably from 1:5 to 1:8.

Preferably, the noble metal particles are evenly distributed among the titanium-containing particles. As used herein, the term “evenly distributed” means the noble metal particles are randomly dispersed among the titanium-containing particles with substantially no agglomeration of the noble metal particles. Preferably, at least 80% of the total number of the noble metal particles are present in a particle having an average diameter less than 15 nm. More preferably, at least 90% of the total number of the noble metal particles are present in a particle having an average diameter less than 15 nm. Even more preferably, at least 95% of the total number of noble metal particles are present in a particle having an average diameter less than 15 nm.

The noble metal particles in the catalyst may be disposed on a surface of a support material. Preferably, the support material is a particle of an oxide material; preferably γ-, δ-, or θ-alumina, silica, magnesia, titania, zirconia, hafnia, vanadia, niobium oxide, tantalum oxide, ceria, yttria, lanthanum oxide or a combination thereof. Preferably, in portions of the catalyst comprising the noble metal, the support has a surface area greater than 10 m²/g, preferably greater than 30 m²/g, preferably greater than 50 m²/g, preferably greater than 100 m₂/g, preferably greater than 120 m²/g. In portions of the catalyst which comprise little or no noble metal, the support may have a surface area less than 50 m²/g, preferably less than m²/g. The average diameter of the support and the average diameter of the final catalyst particle are not significantly different.

Preferably, the aspect ratio of the catalyst particle is no more than 10:1, preferably no more than 5:1, preferably no more than 3:1, preferably no more than 2:1, preferably no more than 1.5:1, preferably no more than 1.1:1. Preferred shapes for the catalyst particle include spheres, cylinders, rectangular solids, rings, multi-lobed shapes (e.g., cloverleaf cross section), shapes having multiple holes and “wagon wheels;” preferably spheres. Irregular shapes may also be used.

The noble metal particles can be dispersed throughout the catalyst or have varying concentration densities, such as, for example, a gradient concentration or layered structure. Preferably, at least 90 wt % of the noble metal(s) is in the outer 70% of catalyst volume (i.e., the volume of an average catalyst particle), preferably the outer 60% of catalyst volume, preferably the outer 50%, preferably the outer 40%, preferably the outer 35%, preferably in the outer 30%, preferably in the outer 25%. Preferably, the outer volume of any particle shape is calculated for a volume having a constant distance from its inner surface to its outer surface (the surface of the particle), measured along a line perpendicular to the outer surface. For example, for a spherical particle the outer x % of volume is a spherical shell whose outer surface is the surface of the particle and whose volume is x % of the volume of the entire sphere. Preferably, at least 95 wt % of the noble metal is in the outer volume of the catalyst, preferably at least 97 wt %, preferably at least 99 wt %. Preferably, at least 90 wt % (preferably at least 95 wt %, preferably at least 97 wt %, preferably at least 99 wt %) of the noble metal(s) is within a distance from the surface that is no more than 30% of the catalyst diameter, preferably no more than 25%, preferably no more than 20%, preferably no more than 15%, preferably no more than 10%, preferably no more than 8%. Distance from the surface is measured along a line which is perpendicular to the surface.

Preferably, the catalyst comprises gold particles and titanium-containing particles on a support material comprising silica. Preferably, the gold particles and titanium-containing particles form an eggshell structure on the support particles. The eggshell layer may have a thickness of 500 microns or less, preferably 250 microns or less, and more preferably 100 microns or less.

Preferably, at least 0.1% by weight of the total weight of the gold particles are exposed on a surface of the catalyst, where the surface includes both the outer surface and pores of the catalyst. As used herein, the term “exposed” means that at least a portion of the gold particle is not covered by another gold particle or titanium-containing particle, i.e., the reactants can directly contact the gold particle. The gold particles may therefore be disposed within a pore of the support material and still be exposed by virtue of the reactant being able to directly contact the gold particle within the pore. More preferably, at least 0.25% by weight of the total weight of the gold particles are exposed on the surface of the catalyst, even more preferably, at least 0.5% by weight of the total weight of the gold particles are exposed on the surface of the catalyst, and still more preferably, at least 1% by weight of the total weight of the gold particles are exposed on the surface of the catalyst.

The catalyst is preferably produced by precipitating the noble metal from an aqueous solution of metal salts in the presence of the support. Suitable noble metal salts may include, but are not limited to, tetrachloroauric acid, sodium aurothiosulfate, sodium aurothiomalate, gold hydroxide, palladium nitrate, palladium chloride and palladium acetate. In one preferred embodiment, the catalyst is produced by an incipient wetness technique in which an aqueous solution of a suitable noble metal precursor salt is added to a porous inorganic oxide such that the pores are filled with the solution and the water is then removed by drying. The resulting material is then converted into a finished catalyst by calcination, reduction, or other treatments known to those skilled in the art to decompose the noble metal salts into metals or metal oxides. Preferably, a C₂-C₁₈ thiol comprising at least one hydroxyl or carboxylic acid substituent is present in the solution. Preferably, the C₂-C₁₈ thiol comprising at least one hydroxyl or carboxylic acid substituent has from 2 to 12 carbon atoms, preferably 2 to 8, preferably 3 to 6. Preferably, the thiol compound comprises no more than 4 total hydroxyl and carboxylic acid groups, preferably no more than 3, preferably no more than 2. Preferably, the thiol compound has no more than 2 thiol groups, preferably no more than one. If the thiol compound comprises carboxylic acid substituents, they may be present in the acid form, conjugate base form or a mixture thereof. The thiol component also may be present either in its thiol (acid) form or its conjugate base (thiolate) form. Especially preferred thiol compounds include thiomalic acid, 3-mercaptopropionic acid, thioglycolic acid, 2-mercaptoethanol and 1-thioglycerol, including their conjugate bases.

In one embodiment of the invention, the catalyst is produced by deposition precipitation in which a porous inorganic oxide is immersed in an aqueous solution containing a suitable noble metal precursor salt and that salt is then made to interact with the surface of the inorganic oxide by adjusting the pH of the solution. The resulting treated solid is then recovered (e.g. by filtration) and then converted into a finished catalyst by calcination, reduction, or other pre-treatments known to those skilled in the art to decompose the noble metal salts into metals or metal oxides.

The catalyst bed may further comprise inert or acidic materials. Preferred inert or acidic materials include, e.g., alumina, clay, glass, silica carbide and quartz. Preferably, the inert or acidic materials located before and/or after the catalyst bed have an average diameter equal to or greater than that of the catalyst, preferably 1 to 30 mm; preferably at least 2 mm; preferably no greater than 30 mm, preferably no greater than 10 mm, preferably no greater than 7 mm.

This invention is useful in a process for producing methyl methacrylate (MMA) which comprises reacting methacrolein with methanol in the presence of an oxygen-containing gas in an oxidative esterification reactor (OER) system containing a catalyst bed.

The catalyst bed, which may comprise a slurry bed or fixed bed, comprises the catalyst particles. The OER system further comprises a liquid phase comprising methacrolein, methanol and MMA and a gaseous phase comprising oxygen. The liquid phase may further comprise byproducts, e.g., methacrolein dimethyl acetal (MDA) and methyl isobutyrate (MIB). Without taking steps to control its formation, MIB may be present in an MMA product stream in amounts in excess of 1 wt % (10,000 ppm) relative to the total weight of MMA, methacrolein and methanol in the product stream exiting the OER system. MIB can be difficult to separate from MMA. Therefore, the present invention seeks to limit the amount of MIB that is formed such that the amount of MIB in the product stream ranges from 0.1 ppm to 5000 ppm, preferably from 0.1 to 4000 ppm, more preferably from 0.1 to 3000 ppm, even more preferably from 0.1 to 2500 ppm, and still more preferably from 0.1 to 2000 ppm.

Preferably, the concentration of methanol entering the OER system is greater than 32 wt % based on the total weight of methanol and methacrolein entering the reactor system. More preferably, the concentration of methanol entering the OER system is greater than 35 wt %, and even more preferably greater than 40 wt % based on the total weight of methanol and methacrolein entering the reactor system. Preferably, the concentration of methanol entering the OER system is less than 75 wt % based on the total weight of methanol and methacrolein entering the reactor system. More preferably, the concentration of methanol entering the OER system is less than 60 wt %, and even more preferably less than 50 wt % based on the total weight of methanol and methacrolein entering the reactor system.

Preferably, the concentration of methanol in the liquid phase product stream exiting the OER system is at least 65 wt % based on the total weight of methanol and methacrolein in the liquid phase product stream exiting the OER system. More preferably, the concentration of methanol in the liquid phase product stream exiting the OER system is at least 70 wt % based on the total weight of methanol and methacrolein in the liquid phase product stream exiting the OER system. Preferably, the concentration of methanol in the liquid phase product stream exiting the OER system is less than 100 wt % based on the total weight of the methanol and methacrolein in the liquid phase product stream exiting the OER system. Preferably, the average concentration of methanol in the OER system (i.e., the arithmetic average of the concentration of methanol entering and exiting the OER system) is greater than 70 wt % based on the average total weight of methanol and methacrolein entering and exiting the OER system (i.e., the arithmetic average of the total weight of methanol and methacrolein entering the OER system and the total weight of methanol and methacrolein exiting the OER system). More preferably, the average concentration of methanol in the OER system is greater than 75 wt % based on the average total weight of methanol and methacrolein entering and exiting the OER system.

It is preferred that the average weight ratio of methanol to methacrolein in the OER system ranges from 20:1 to 2:1, where the average weight ratio is based on the average concentration of methanol entering and exiting the OER system and the average concentration of methacrolein entering and exiting the OER system.

One example of an OER system comprises a multizone or multi-reactor system. In a first zone or reactor, the average concentration of methanol in the first zone or reactor ranges from 50 wt % to 80 wt % based on the average total amount of methanol and methacrolein entering and exiting the first zone or reactor. The final zone or reactor has an average methanol concentration ranging from 80 wt % to 100 wt % based on the average total amount of methanol and methacrolein entering and exiting the final zone or reactor. Between the first zone or reactor and the final zone or reactor, the reactor mixture may be cooled and/or additional oxygen may be added, such as, for example, by adding air to a gas phase entering the final zone or reactor.

Preferably, oxygen concentration in a gas stream exiting the OER system is at least 1 mol %, more preferably at least 2 mol %, even more preferably at least 2.5 mol %, still more preferably at least 3 mol %, yet more preferably at least 3.5 mol %, even yet more preferably at least 4 mol %, and most preferably at least 4.5 mol %, based on the total volume of the gas stream exiting the OER system. Preferably, the oxygen concentration in a gas stream exiting the OER system is no more than 7.5 mol %, preferably no more than 7.25 mol %, preferably no more than 7 mol %, based on the total amount of the gas stream exiting the OER system.

Preferably, the liquid phase in the OER system is at a temperature from 40 to 120° C.; preferably at least 50° C., and preferably at least 55° C. The temperature of the liquid phase in the OER system is preferably no more than 110° C., and preferably no more than 100° C. When the OER system comprises more than one reactor and/or more than one zone, the temperature in each reactor and/or zone may be the same or different. For example, a reaction mixture exiting a reactor or zone may be cooled prior to entering the next reactor or zone.

Preferably, the catalyst bed in the OER system is at a pressure from 1 to 150 bar (100 to 15000 kPa). Without wishing to be limited by theory, it is believed that operating the OER system at increased pressure will lower the amount of MIB present in the product stream by increasing the amount of oxygen present in the liquid phase. Therefore, the pressure in the catalyst bed of the OER system may be at least 10 bar, preferably at least 20 bar, preferably at least 30 bar, preferably at least 40 bar, or preferably at least 60 bar. For example, the pressure in the catalyst bed of the OER system may be at least 100 bar. When the OER system comprises more than one reactor and/or zone, the pressure in each reactor and/or zone may be the same or different.

The heterogeneous noble metal-containing catalyst in the OER system may be present in an amount ranging from 0.02 kg to 2 kg of catalyst for every gram-mole of methyl methacrylate exiting the reactor system over the course of 1 hour. Preferably, the heterogeneous noble metal-containing catalyst in the OER system is present in an amount of at least 0.02 kg to 0.5 kg of catalyst, for every gram-mole of methyl methacrylate exiting the reactor system over the course of 1 hour. Preferably, the heterogeneous noble metal-containing catalyst in the OER system is present in an amount of less than 0.4 kg of catalyst, more preferably less than 0.3 kg of catalyst, still more preferably less than 0.25 kg of catalyst, and even more preferably less than 0.2 kg of catalyst for every gram-mole of methyl methacrylate exiting the reactor system over the course of 1 hour.

The amount of methyl methacrylate exiting the reactor is dependent on the conversion of methacrolein in the OER system. For example, at 50% conversion of methacrolein entering the OER system, 2 moles of methacrolein would be required for every mole of methyl methacrylate produced. In this example, the heterogeneous noble metal-containing catalyst in the OER system may be present in an amount ranging from 0.01 to 1 kg of catalyst for every gram-mole of methacrolein entering the reactor system over the course of 1 hour. At 25% conversion of methacrolein entering the OER system, 4 moles of methacrolein would be required for every mole of methyl methacrylate produced, and the heterogeneous noble metal-containing catalyst in the OER system may be present in an amount ranging from 0.005 to 0.5 kg of catalyst for every gram-mole of methacrolein entering the reactor system over the course of 1 hour. At 75% conversion of methacrolein entering the OER system, 1.33 moles of methacrolein would be required for every mole of methyl methacrylate produced, and the heterogeneous noble metal-containing catalyst in the OER system may be present in an amount ranging from 0.015 to 1.5 kg of catalyst for every gram-mole of methacrolein entering the reactor system over the course of 1 hour. Disregarding any external recycle streams, the OER system preferably exhibits at least 25% conversion of methacrolein to methyl methacrylate, more preferably at least 35% conversion, and even more preferably at least 40% conversion of methacrolein to methyl methacrylate in the OER system. Addition of an external recycle stream that recycles unreacted methacrolein to the OER system can also be used to improve the overall conversion efficiency of the process.

When the noble metal-containing catalyst comprises gold, the gold may be present in an amount ranging from 0.0001 kg to 0.1 kg for every gram-mole of MMA exiting the reactor system over the course of 1 hour. Preferably, the gold is present in an amount of at least 0.0001 kg to 0.005 kg for every gram-mole of MMA exiting the reactor system over the course of 1 hour. Preferably, the gold is present in an amount less than 0.004 kg for every gram-mole of MMA exiting the reactor system over the course of 1 hour.

In terms of the amount of heterogeneous noble metal-containing catalyst in the OER system with respect to the amount of methacrolein entering the reactor system, at 50% conversion of methacrolein entering the OER system, the gold in the heterogeneous noble metal-containing catalyst in the OER system may be present in an amount ranging from 0.00005 to 0.05 kg of gold for every gram-mole of methacrolein entering the reactor system over the course of 1 hour. At 25% conversion of methacrolein entering the OER system, the gold in the heterogeneous noble metal-containing catalyst in the OER system may be present in an amount ranging from 0.000025 to 0.025 kg of catalyst for every gram-mole of methacrolein entering the reactor system over the course of 1 hour. At 75% conversion of methacrolein entering the OER system, the gold in the heterogeneous noble metal-containing catalyst in the OER system may be present in an amount ranging from 0.000075 to 0.075 kg of catalyst for every gram-mole of methacrolein entering the reactor system over the course of 1 hour.

The pH in the catalyst bed may range from 2 to 10. Some catalysts may be deactivated in acidic conditions. Therefore, when the catalyst is not acid resistant, the pH in the catalyst bed is from 4 to 10; preferably at least 5, preferably at least 5.5; preferably no greater than 9, preferably no greater than 8, preferably no greater than 7.5.

To increase the pH in the reactor system, a base material may be added. The present inventors have discovered that Arrhenius bases (i.e., a compound that dissociates in water to form hydroxide ions, such as hydroxides of alkali and alkaline earth metals) can lead to the detrimental accumulation of salts within the catalyst bed. Therefore, the OER system does not comprise an Arrhenius base. If a base is used to adjust the pH, the base material is preferably a Lewis base (i.e., a compound capable of donating a pair of electrons) or Bronsted-Lowry base (i.e., a compound capable of accepting a proton) that is not also classified as an Arrhenius base. In other words, the base material may be selected from a base that does not comprise a hydroxide of an alkali or alkaline earth metal. Examples of Lewis bases include, but are not limited to, amines, sulfates, and phosphines. Examples of Bronsted-Lowry bases include, but are not limited to, halides, nitrates, nitrites, chlorites, chlorates, etc. Ammonia can be either a Lewis base or a Bronsted-Lowry base.

The present inventors have discovered that high localized concentrations of base materials in the reactor system can cause the formation of unwanted Michael adduct as byproducts. Therefore, to aid in the minimization of the formation of Michael adducts, the base material is preferably mixed with at least one other material prior to entering the reactor system. Preferably, the base material is introduced at a position external to the reactor system and mixed with one or more reactants or diluents to form a base-containing stream. For example, the base material may be mixed with methanol, water, or a non-reactive solvent, i.e., a solvent that does not negatively impact the formation of methyl methacrylate in the reactor system. The position external to the reactor system may be a mixing vessel. Alternatively, the position external to the reactor may be a line through which components travel to the reactor system, such as a feed line or a recycle line, in which sufficient mixing occurs, such as by turbulent flow, baffles, jet mixer, or other mixing method.

Preferably, the amount of the base material in the base-containing stream is 50 wt % or less based on the total weight of the base-containing stream, preferably 25 wt % or less, preferably 20 wt % or less, preferably 15 wt % or less, preferably 10 wt % or less, preferably 5 wt % or less, or preferably 1 wt % or less. The base material is preferably diluted by a factor of less than 1:2, such as, less than 1:3, less than 1:4, less than 1:5, less than 1:10, less than 1:20, or less than 1:100, relative to the total weight of the base-containing stream prior to entering the reactor system.

Preferably, the base-containing stream is sufficiently mixed to avoid localized spikes in the concentration of the base material within the base-containing stream before it is added to the reactor system. For example, it is preferred that the base-containing stream reach at least 95% degree of homogeneity, i.e., variations in the concentration of the base material deviate within +/−5% of the average concentration of base material for the base-containing stream prior to entering the reactor system. Preferably, the base-containing stream reaches 95% degree of homogeneity within 4 minutes of introduction of the base material, more preferably within 2 minutes, and even more preferably within 1 minute of introduction of the base material.

For a mixing vessel, the time required for an additive to reach a 95% degree of homogeneity is defined at Θ₉₅, which can be calculated by the method disclosed by Grenville and Nienow, The Handbook of Industrial Mixing, Pages 507-509, which gives the following expression for a stirred tank in turbulent flow:

$${θ}_{95} = 5.2 \frac{{T}^{1.5} {H}^{0.5}}{{D}^{2} {N}_{P}^{1 / 3} N}$$

where T is the tank diameter, H is the liquid height, D is the impeller diameter, Np is the characteristic power number of the impeller(s), and N is the impeller speed. Similar expressions exist for static mixers, jet mixed vessels, etc.

Preferably, no base material is added to the reactor system, either internal or external to the reactor system. Preferably, when no base material is added to the reactor system, the noble metal-containing catalyst comprises an acid-resistant catalyst such as a catalyst comprised of gold and titanium-containing particles. Operating the OER system in the absence of a base material may provide several advantages. One advantage is the increased selectivity and space time yield (STY) due to lower production of Michael adducts. Another advantage is the reduction in cost due to the reduced cost to treat aqueous waste. Aqueous waste exiting an oxidative esterification process in which a base material was used can produce large quantities of inorganic salts, which can be difficult or impossible to treat with biological water treatment processes. This in turn, may require the use of other waste treatment process, such as incineration.

The OER typically produces a liquid product stream comprising MMA, along with methacrylic acid and unreacted methanol. Preferably, the reaction products are fed to a methanol recovery distillation column which provides an overhead stream rich in methanol and methacrolein; preferably this stream is recycled back to the OER. The bottoms stream from the methanol recovery distillation column comprises MMA, MIB, MDA, methacrylic acid, salts and water. MDA is preferably hydrolyzed in a medium comprising MMA, MDA, methacrylic acid, salts and water. MDA may be hydrolyzed in the bottoms stream from the methanol recovery distillation column. This hydrolysis may take place within the methanol recovery column. The bottoms stream from the methanol recovery distillation column may be sent to a separate acetal hydrolysis reactor for additional MDA hydrolysis. Alternatively, MDA may be hydrolyzed in a separate acetal hydrolysis reactor after the organic phase separated from the methanol recovery bottoms stream. It may be necessary to add water to the organic phase to ensure that there is sufficient water for the MDA hydrolysis; these amounts may be determined easily from the composition of the organic phase. An acid stream may also be added to the hydrolysis reactor to ensure adequate MDA removal. The product of the MDA hydrolysis reactor is phase separated and the organic phase passes through one or more distillation columns to produce MMA product and light and/or heavy byproducts.

The methacrolein used in the oxidative esterification reaction is preferably produced by either an aldol condensation or Mannich condensation. Preferably, the methacrolein is formed by the Mannich condensation of propionaldehyde and formaldehyde in the presence of a suitable catalyst. The molar ratio of propionaldehyde to formaldehyde may range from 1:20 to 20:1, preferably from 1:1.5 to 1.5:1, more preferably 1:1.25 to 1.25:1, and even more preferably from 1:1.1 to 1.1:1.

Examples of catalysts that may be used in a Mannich condensation process include, for example, amine-acid catalysts. Acids of the amine-acid catalysts may include, but are not limited to, inorganic acids (e.g., sulfuric acid and phosphoric acid) and organic mono-, di-, or polycarboxylic acids (e.g., aliphatic C₁-C₁₀ monocarboxylic acids, C₂-C₁₀ dicarboxylic acids, C₂-C₁₀ polycarboxylic acids). Amines of the amine-acid catalysts may include, but are not limited to compounds of formula NHR¹R², where R¹ and R² are each independently C₁-C₁₀ alkyl, which are optionally substituted with an ether, hydroxyl, secondary amino or tertiary amino group, or R¹ and R², together with the adjacent nitrogen, may form a C₅-C₇ heterocyclic ring, optionally containing a further nitrogen atom and/or an oxygen atom, and which are optionally substituted by a C₁-C₄ alkyl or C₁-C₄ hydroxyalkyl.

The Mannich condensation reaction is preferably carried out in the liquid phase by reacting propionaldehyde, formaldehyde, and methanol in the presence of an amine-acid catalyst in a reactor at a temperature of at least 20° C. and at a pressure greater than 1 bar. The temperature of the reactor may range from 20° C. to 220° C., preferably from 80° C. to 220° C., and more preferably from 120° C. to 220° C. The pressure of the reactor may range from greater than 1 bar to 150 bar.

Inhibitors can be added to the reactor to prevent the formation of unwanted products. For example, 4-hydroxy-2,2,6,6-tetramethylpiperidin-1-oxyl (4-hydroxy-TEMPO) can be added to the reactor.

The propionaldehyde used to prepare the methacrolein can be prepared by the hydroformylation of ethylene. The hydroformylation process is known in the art, and is disclosed, for example, in U.S. Pat. Nos. 4,427,486, 5,087,763, 4,716,250, 4,731,486, and 5,288,916. The hydroformylation of ethylene to propionaldehyde comprises contacting ethylene with carbon monoxide and hydrogen in the presence of a hydroformylation catalyst. Examples of hydroformylation catalysts include, for example, metal-organophosphorus ligand complexes, such as organophosphines, organophosphites, and organophosphoramidites. The ratio of carbon monoxide to hydrogen may range from 1:10 to 100:1, preferably from 1:10 to 10:1. The hydroformylation process may be conducted at a temperature ranging from −25° C. to 200° C., preferably from 50° C. to 120° C.

Ethylene used to prepare propionaldehyde may be prepared from the dehydration of ethanol. For example, ethylene can be prepared by the acid-catalyzed dehydration of ethanol. Ethanol dehydration is known in the art and is disclosed, for example, in U.S. Pat. No. 9,249,066. Preferably, ethanol is sourced from renewable resources, such as plant materials or biomass, as opposed to ethanol prepared from petroleum based sources. Using bio-resourced ethanol alone in the process for producing MMA can result in up to 40% of the carbon atoms of the MMA (i.e., 2 of the 5 carbon atoms in the MMA) coming from renewable resources.

To further increase the renewable carbon content in the MMA, additional starting materials can also be prepared from renewable resources. For example, formaldehyde can be prepared from syngas, where the syngas can be prepared from biomass. Carbon monoxide, which can also be used in the preparation of propionaldehyde, can also be prepared from renewable resources, as disclosed by Li et al., ACS Nano, 2020, 14, 4, 4905-4915. Using these additional bio-resourced can further increase the amount of renewable carbon.

Alternatively, starting materials to produce the MMA can be prepared from recycled materials. For example, recycled carbon dioxide can be used to produce methanol, and the methanol can be used to produce formaldehyde.

Preferably, at least 40% of the carbon atoms in the MMA are derived from renewable or recycled content, more preferably at least 60%, even more preferably at least 80%, and still more preferably 100%.

1. A process for producing methyl methacrylate by oxidative esterification, the process comprising:
reacting methanol and methacrolein in a reactor system comprising an oxygen-containing gas and a heterogeneous noble metal-containing catalyst to produce methyl methacrylate,
wherein no Arrhenius base is added to the reactor system.

2. The process of claim 1, wherein a base material comprising a Lewis base is added to the reactor system to maintain a pH between 4 and 10.

3. The process of claim 2, wherein the base material is added directly or indirectly to the reactor system.

4. The process of claim 3, wherein the base material is added indirectly to the reactor system by mixing with one or more reactants to form a base-containing stream that is subsequently added to the reactor system.

5. The process of claim 1, wherein no base is added to the reactor system.

6. The process of claim 5, wherein a pH of the reactor system ranges from 2 to 7.

7. The process of claim 1, wherein the reactor system comprises a fluidized bed, a fixed bed, a trickle bed, a packed bubble column, or a stirred bed reactor.

8. The process of claim 1, wherein the heterogeneous noble metal-containing catalyst is in the form of a slurry or fixed bed.

9. The process of claim 1, wherein a portion of a product stream exiting the reactor system is recycled back to the reactor system without adding a base material.

10. The process of claim 9, wherein the portion of the product stream recycled back to the reactor system is distilled to remove methyl methacrylate and/or heavy materials and/or light materials before entering the reactor system.

11. The process of claim 1, wherein the heterogeneous noble metal-containing catalyst comprises gold.

12. The process of claim 1, wherein a gas phase stream exiting the reactor system comprises oxygen in an amount ranging from 1 mol % to 7.5 mol % based on the total amount of the gas phase.

13. The process of claim 1, wherein the reactor system is maintained at a temperature ranging from 40° C. to 120° C.

14. The process of claim 1, wherein the reactor system is maintained at a pressure ranging from 0 psig to 300 psig.

15. The process of claim 1, wherein the molar ratio of methanol to methacrolein entering the reactor system ranges from 1:100 to 1000:1.

16. The process of claim 14, wherein the molar ratio of methanol to methacrolein entering the reactor system ranges from 0.1:1 to 10:1.
